(19) United States
(12) Patent Application Publication
(10) Pub. No.: US 2018/0140609 A1
(43) Pub. Date: May 24, 2018

(54) USE OF (4-HYDROXY-2-METHYL-1,1-DIOXIDO-2H-BENZO[E][1,2]THIAZINE-3-YL)(NAPHTHALENE-2-YL) METHANONE IN THE PREVENTION AND/OR TREATMENT OF NON-ALCOHOLIC STEATOHEPATITIS

(71) Applicant: PIERRE FABRE MEDICAMENT, Boulogne-Billancourt (FR)

(72) Inventors: Marie LAMOTHE, Castres (FR); Didier JUNQUERO, Castres (FR); Bruno LE GRAND, Teyssode (FR)

(73) Assignee: PIERRE FABRE MEDICAMENT, Boulogne-Billancourt (FR)

(21) Appl. No.: 15/568,085

(22) PCT Filed: Apr. 20, 2016
(86) PCT No.: PCT/EP2016/058760

§ 371 Date: Oct. 20, 2017

(30) Foreign Application Priority Data
Apr. 21, 2015 (FR) 1553574

(51) Int. Cl. A61K 31/5415

(52) U.S. Cl. CPC A61K 31/5415; A61K 45/06

(57) ABSTRACT

The present invention relates to (4-hydroxy-2-methyl-1,1-dioxido-2H-benzo[e][1,2]thiazine-3-yl)(naphthalen-2-yl)methanone or one of the salts thereof, pharmaceutically acceptable for use in the prevention and/or treatment of hepatic steatosis, including non-alcoholic steatohepatitis or one of the complications of same.

The present invention relates to (4-hydroxy-2-methyl-1,1-dioxido-2H-benzo[e][1,2]thiazin-3-yl)(naphthalen-2-yl)methanone, or a pharmaceutically acceptable salt thereof, for use in the prevention and/or treatment of fatty liver (hepatic steatosis), including non-alcoholic steatohepatitis or a complication thereof.

(4-Hydroxy-2-methyl-1,1-dioxido-2H-benzo[e][1,2]thiazin-3-yl)(naphthalen-2-yl)methanone represented by the formula:

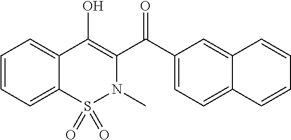 [SMILES: CN1C(C(=O)c2ccc3ccccc3c2)=C(O)c2ccccc2S1(=O)=O]

the pharmaceutically acceptable salts thereof and the use of same as both curative and preventive treatment for type 2 diabetes, obesity, dyslipidemia, arterial hypertension, atherosclerosis and clinical pathologies resulting therefrom such as coronary events, cerebrovascular accidents or arteritis of the lower limbs, hyperglycemia, glucose intolerance, insulin resistance, hypertriglyceridemia, hypercholesterolemia, restenosis, pancreatitis, retinopathy, nephropathy, neuropathy, certain types of cancer and glaucoma are described in patent application WO 2010/100139.

Although viral hepatitis and alcoholic liver disease are significant worldwide, they do not represent all liver diseases, not even the most important. Over the past couple of decades, it has become increasingly clear that non-alcoholic fatty liver disease (NAFLD) and non-alcoholic steatohepatitis (NASH) are now the number one cause of liver disease in the West.

Fatty liver results from the accumulation of fats in hepatocytes. Fatty compounds slowly accumulate in the liver when the amount of accumulated fats exceeds that which the body can process. A person has fatty liver when fat makes up at least 5% of the liver. Simple fatty liver can be an entirely benign state and usually does not cause liver damage. However, once there is accumulation of simple fats, the liver becomes vulnerable to subsequent damage that can cause inflammation and scarring. Fatty liver is becoming increasingly common in children mainly because of an alarming increase in child obesity.

NASH is a highly-prevalent chronic liver disease in which steatosis is associated with histological lesions of liver cells and lobular inflammation (LaBrecque et al., World Gastroenterology Organisation Global Guidelines, J. Clin. Gastroenterol. 48, 467-473, 2014) and potentially fibrosis. NASH is often associated with an increased risk of cardiovascular disease and notably cerebrovascular accidents.

The pathogenesis of NASH is multifactorial; it includes various physiopathological mechanisms leading to peripheral and hepatic insulin resistance, disorders of fatty acid metabolism, oxidative stress and cellular fibroproliferation (Cusi, Gastroenterol. 142, 711-725, 2012). To date, however, NASH diagnosed by liver biopsy is not always associated with diabetes or dyslipidemia. The complexity and multimodality of this pathology explain the absence of established and recognized therapeutic treatment to date and the ongoing clinical trials and the multiplicity of mechanistic approaches. However, medical treatment with a bile acid derivative having antioxidant and anti-inflammatory properties may be envisaged (Coskun et al., Eur. J. Gastroenterol. Hepatol. 27, 142-149, 2015).

Glucocorticoids—cortisol in humans—are ubiquitous hormones which play a preponderant role in the regulation of energy metabolism. They promote gluconeogenesis and inhibit insulin secretion and peripheral glucose reuptake. 11β-Hydroxysteroid dehydrogenases (11β-HSDs) regulate glucocorticoid levels in certain target tissues—such as liver, adipose tissue, kidney and brain—by ensuring the interconversion of cortisone and cortisol (Chapman et al., Physiol. Rev. 93, 1139-1206, 2013; Morgan et al., Proc. Natl. Acad. Sci. 111, E2482-E2491, 2014; Gathercole et al., Endocrine Rev. 34, 525-555, 2013). In adipose tissue, a local increase in cortisol can lead to an exacerbated increase in lipolysis, further causing a rise in free fatty acids in the blood and an increased portal influx of lipids in the liver; this mechanism, called “lipotoxicity,” causes fatty liver, stimulation of de novo lipogenesis that can develop into NASH, and gluconeogenesis (Ahmed et al., PLOS ONE 7, e29531, 2012). Beyond this “peripheral adipose tissue-liver” interaction, the regeneration of intrahepatic cortisol via 11β-hydroxysteroid dehydrogenase type 1 can also cause gluconeogenesis and accentuate peripheral insulin resistance. Very recently it was shown that a reduction of 11β-hydroxysteroid dehydrogenase type 1 activity in mice causes an increase in the circulating and intrahepatic bile acids (Penno et al., Mol. Metab. 3, 554-546, 2014) that play a key role in lipid homeostasis and liver physiopathology (Porez et al., J. Lipid Res. 53, 1723-1737, 2012).

The significance of the inhibition of 11β-hydroxysteroid dehydrogenase type 1 was studied clinically in fatty liver disease in overweight but non-diabetic subjects (Stefan et al., Lancet Diabetes Endocrinol. 2, 406-416, 2014). In that study, no improvement in peripheral insulin sensitivity was reported, suggesting that this 11β-hydroxysteroid dehydrogenase type 1 inhibitor acts by another mechanism of action. Nevertheless, with other compounds the increase in insulin sensitivity was clearly shown (Rosenstock et al., Diabetes Care 33, 1516-1522, 2010), which underlines the major differences in activity profile within the same therapeutic class and thus makes it impossible to predict the tissue distribution of one compound or another.

Thus, the importance of inhibition of 11β-hydroxysteroid dehydrogenase type 1 in all metabolically-active target tissues, and notably in peripheral adipose tissue due to its interactions with the liver via lipolysis, appears to be a key factor of success for an inhibitory molecule in order to reduce hepatic lipotoxicity and to treat NASH and associated pathologies.

The inventors unexpectedly discovered that (4-hydroxy-2-methyl-1,1-dioxido-2H-benzo[e][1,2]thiazin-3-yl)(naphthalen-2-yl)methanone, or a pharmaceutically acceptable salt thereof, in particular the potassium salt, could be used to treat fatty liver, notably NASH or a complication thereof.

In the present invention, the term “pharmaceutically acceptable” refers to molecular entities and compositions that produce no adverse or allergic effect or other undesirable reaction when they are administered to a human. When used herein, the term “pharmaceutically acceptable excipient” includes any diluent, adjuvant or excipient, such as preservatives, fillers, disintegrants, wetting agents, emulsifiers, dispersants, antibacterials or antifungals, or indeed agents for delaying intestinal and digestive absorption and resorption. The use of these media or vehicles is well-known to a person skilled in the art.

The pharmaceutically acceptable salts for therapeutic use of the compound of the present invention include the conventional non-toxic salts of the compound of the invention such as those formed from organic or inorganic bases. By way of example, mention may be made of the salts derived from inorganic bases such as sodium hydroxide, potassium hydroxide or calcium hydroxide and the salts derived from organic bases such as lysine or arginine. These salts may be synthesized from the compound of the invention containing an acid moiety and the corresponding bases according to conventional chemical methods.

The proton of the hydroxyl function present in (4-hydroxy-2-methyl-1,1-dioxido-2H-benzo[e][1,2]thiazin-3-yl)(naphthalen-2-yl)methanone (Compound 1) is sufficiently acidic to be able to be salified according to the techniques known to a person skilled in the art. Thus, by way of example, potassium 3-(2-naphthoyl)-2-methyl-2H-benzo[e][1,2]thiazin-4-olate 1,1-dioxide may be obtained after dissolution of Compound 1 in a 50:50 mixture of ethanol and dichloromethane then addition of an equivalent of 1 M aqueous potassium hydroxide solution. The present invention more particularly relates to this particular potassium salt. A second example is sodium 3-(2-naphthoyl)-2-methyl-2H-benzo[e][1,2]thiazin-4-olate 1,1-dioxide, obtained as before by replacing the aqueous potassium hydroxide solution with aqueous sodium hydroxide solution.

The acceptable solvates for therapeutic use of the compound of the present invention include conventional solvates such as those formed during the last step of preparing the compound of the invention due to the presence of solvents. By way of example, mention may be made of solvates due to the presence of water or ethanol.

According to the present invention, the potential complications of NASH are liver fibrosis, cirrhosis, liver failure and hepatocellular carcinoma.

Liver fibrosis is the common result of chronic liver disease, characterized by abnormally high accumulation of extracellular matrix components in the liver parenchyma. Its progression may lead to cirrhosis.

Cirrhosis may result from various causes, such as chronic alcohol consumption, fat accumulation in the liver and autoimmune diseases. It is defined according to morphological criteria of fibrosis and of transformation of the normal architecture of the liver into structurally abnormal nodules. These abnormalities are accompanied by disruptions of liver function.

Liver failure, sometimes referred to as fulminant hepatitis, is a serious acute deterioration of hepatocellular function. The initial consequences are major disorders of hemostasis which lead to a risk of multiorgan hemorrhage. Its prognosis is extremely harsh.

Hepatocellular carcinoma is a primary liver cancer. It usually develops on cirrhosis. For patients with risk factors for hepatocellular carcinoma (cirrhosis, NASH), increased monitoring of biological and morphological parameters is now employed to detect the disease at an earlier stage.

(4-Hydroxy-2-methyl-1,1-dioxido-2H-benzo[e][1,2]thiazin-3-yl)(naphthalen-2-yl)methanone is an inhibitor of 11β-hydroxysteroid dehydrogenase type 1.

The invention relates to the use of (4-hydroxy-2-methyl-1,1-dioxido-2H-benzo[e][1,2]thiazin-3-yl)(naphthalen-2-yl)methanone, or a pharmaceutically acceptable salt thereof, as a medicinal product in the prevention and/or treatment of fatty liver.

The invention also relates to the use of (4-hydroxy-2-methyl-1,1-dioxido-2H-benzo[e][1,2]thiazin-3-yl)(naphthalen-2-yl)methanone, or a pharmaceutically acceptable salt thereof, as a medicinal product in the prevention and/or treatment of non-alcoholic steatohepatitis or a complication thereof, such as for example liver fibrosis, cirrhosis, liver failure or hepatocellular carcinoma.

It is important to note that the use of (4-hydroxy-2-methyl-1,1-dioxido-2H-benzo[e][1,2]thiazin-3-yl)(naphthalen-2-yl)methanone, or a pharmaceutically acceptable salt thereof, is also suitable as a medicinal product in the prevention and/or treatment of fatty liver or steatohepatitis, residual after alcohol withdrawal.

The present invention further relates to a pharmaceutical composition comprising (4-hydroxy-2-methyl-1,1-dioxido-2H-benzo[e][1,2]thiazin-3-yl)(naphthalen-2-yl)methanone, or a pharmaceutically acceptable salt thereof, as active ingredient and at least one pharmaceutically acceptable excipient, for use as a medicinal product in the prevention and/or treatment of fatty liver, notably in patients with non-alcoholic steatohepatitis or a complication thereof such as, for example, liver fibrosis, cirrhosis, liver failure or hepatocellular carcinoma.

The composition according to the invention may be formulated and/or administered with one or more other active agents, such as an agent active in type 2 diabetes and/or dyslipidemia.

In a preferred manner, the composition according to the invention may be formulated and/or administered in combination with insulin-resistance medicinal products, such as biguanides, for example metformin, insulin-secretors such as hypoglycemic sulfamides, glinides, GLP-1 analogs, gliptins, or alpha-glucosidase inhibitors.

In another preferred manner, the composition according to the invention may be formulated and/or administered in combination with statins or HMG-CoA reductase inhibitors, inhibitors of intestinal cholesterol absorption, such as ezetimibe, fibrates, ion-exchange resins or nicotinic acid.

In a particularly preferred manner, the composition according to the invention may be formulated and/or administered in combination with a monoclonal antibody against PCSK9, such as for example alirocumab or evolocumab.

The pharmaceutical compositions according to the present invention may be formulated for administration to humans. The compositions according to the invention may be administered orally, sublingually, subcutaneously, intramuscularly, intravenously, transdermally, locally, rectally or intranasally. In this case the active ingredient may be administered in single-unit dosage forms, mixed with conventional pharmaceutical carriers, to humans. The suitable single-unit dosage forms include oral dosage forms such as tablets, capsules, powders, granules and oral solutions or suspensions; sublingual and buccal dosage forms; subcutaneous or transdermal, topical, intramuscular, intravenous, intranasal or intraocular dosage forms; or rectal dosage forms.

When a solid composition in tablet form is prepared, the main active ingredient is mixed with a pharmaceutical vehicle such as gelatin, starch, lactose, magnesium stearate, talc, gum arabic, silica or the like. The tablets may be coated with sucrose or other suitable materials or they may be treated such that they have extended or delayed activity and that they continuously release a predetermined amount of active ingredient.

Capsules may be obtained by mixing the active ingredient with at least one formulation excipient and by pouring the mixture obtained into soft or hard capsules.

A preparation in syrup or elixir form may contain the active ingredient together with a sweetener, an antiseptic, as well as a flavoring agent and a suitable colorant.

Water-dispersible powders or granules may contain the active ingredient mixed with dispersants or wetting agents, or suspension agents, just as with flavor correctors or sweeteners.

For rectal administration use is made of suppositories, which are prepared with binders that melt at rectal temperature, for example cocoa butter or polyethylene glycols.

For parenteral (intravenous, intramuscular, intradermal, subcutaneous), intranasal or intraocular administration, use is made of aqueous suspensions, isotonic saline solutions or sterile injectable solutions containing pharmacologically compatible dispersants and/or wetting agents.

The active ingredient may also be formulated as microcapsules, optionally with one or more additional carriers.

Advantageously, the pharmaceutical composition according to the present invention is intended for oral administration.

The dosages of (4-hydroxy-2-methyl-1,1-dioxido-2H-benzo[e][1,2]thiazin-3-yl)(naphthalen-2-yl)methanone, or a pharmaceutically acceptable salt thereof, in the compositions of the invention may be adjusted in order to have the amount of substance that is effective in obtaining the desired therapeutic response for a composition specific to the method of administration. The effective dose of the compound of the invention varies according to many parameters, such as, for example, the chosen administration route; the weight, age, sex and sensitivity of the individual to be treated; and the nature of the pathology. Consequently, the optimal dosing regimen should be determined by a person skilled in the art as a function of the parameters deemed relevant. Although the effective doses may vary in large proportions, daily doses may be between 1 mg and 2000 mg each 24 hours, and preferably between 50 and 1000 mg, for an adult weighing 70 kg on average, in one or more administrations.

The following examples illustrate the invention without limiting its scope.

### EXAMPLE1

### Inhibition of Human 11β-Hydroxysteroid Dehydrogenase Type 1 (11β-HSD 1); In Vitro Test on Primary Human Differentiated Adipocytes in Culture

Inhibition of human 11β-HSD 1 enzyme is evaluated on primary human adipocytes in culture (ZenBio).

### Protocol:

Preadipocytes are thawed and their viability is confirmed. The preadipocytes are then placed in 96-well microplates in a specific preadipocyte medium (PM-1) provided by ZenBio; the plates are incubated at 37° C. with 5% CO₂. One day or more after the cells are confluent, PM-1 is replaced with a more specific differentiation medium (DM), also provided by ZenBio, containing isobutylmethylxanthine, insulin, dexamethasone and a PPAR agonist. The cells will differentiate into adipocytes after a minimum of 7 days. Next, the mature adipocytes are maintained for 4 to 6 days in adipocyte maintenance medium (AM). Then, in the presence of serum treated with dextran-coated charcoal, the cells are placed in steroid-deficient conditions for 48 h, and are pretreated with the inhibitors to be tested or their carrier (0.1% DMSO) for 1 h at 37° C. before the pulse with cortisone. Tritiated cortisone is added for 4 hours to reach a final concentration of 20 nM. The cortisol concentration is quantified from the cell supernatant using SPA technology. EC₅₀ values are obtained with the SigmaPlot v.11 software, four parameter logistic equation; the reported values come from three different experiments carried out on three different donors.

### Inhibitors Tested:

Compound 1K: potassium salt of Compound 1, potassium 3-(2-naphthoyl)-2-methyl-2H-benzo[e][1,2]thiazin-4-olate 1,1-dioxide;

Compound 2: 3′-(4-hydroxy-2-methyl-1,1-dioxido-2H-benzo[e][1,2]thiazine-3-carbonyl)-[1,1′-biphenyl]-4-carbonitrile.

### Results

| | EC₅₀ | Eₘₐₓ |
|---|---|---|
| | (nM) | (% inhibition at 10 μM) |
| Compound 1K | 58 | 97 |
| Compound 2 | ~10 | 99 |

Compounds 1K and 2 are powerful inhibitors of 11β-HSD type 1 in primary human adipocytes in culture.

### EXAMPLE 2

### Evaluation of Plasma Bioactivity After Single Oral Administration of Various 11β-HSD Type 1 Inhibitor Compounds in C57BL/6N Mice (Bioavailability/Power/Efficacy)

### Protocol

The evaluation is made in on 4- to 6-week-old non-fasted male C57BL/6N strain mice. The compounds to be tested or the vehicle (0.5% methylcellulose in water, at 10 ml/kg) are administered orally; n=3 mice per treatment.

Blood samples are collected 1 h and 4 h after administration; the tubes are centrifuged to obtain plasma and frozen at −70° C. until bioanalysis. Plasma bioactivity (2% final volume) is analyzed by using the inhibition of human 11β-HSD type 1 as a detection system (SPA technology) for each compound at each dose tested. Percent inhibition (versus mice treated with carrier) is calculated for each dose.

### Compounds Tested:

Compound 1: (4-hydroxy-2-methyl-1,1-dioxido-2H-benzo[e][1,2]thiazin-3-yl)(naphthalen-2-yl)methanone;

Compound 1K: potassium salt of Compound 1, potassium 3-(2-naphthoyl)-2-methyl-2H-benzo[e][1,2]thiazin-4-olate 1,1-dioxide;

Compound 2: 3′-(4-hydroxy-2-methyl-1,1-dioxido-2H-benzo[e][1,2]thiazine-3-carbonyl)-[1,1′-biphenyl]-4-carbonitrile, described in patent WO 2010/100139;

Compound 2K: potassium salt of Compound 2, potassium 3-(4′-cyanobiphenyl-4-carbonyl)-2-methyl-2H-benzo[e][1,2]thiazin-4-olate 1,1-dioxide;

Compound 3: reference compound, (3-(1-adamantyl)-6,7,8,9-tetrahydro-5H-[1,2,4]triazolo[4,3-a]azepine, described in patent application US2005/0070720.

### Results

| | % inhibition at 1 h | | | | | % inhibition at 4 h | | | | |
|---|---|---|---|---|---|---|---|---|---|---|
| | Doses (mg/kg) | | | | | Doses (mg/kg) | | | | |
| Compounds | 0.63 | 2.5 | 10 | 40 | 80 | 0.63 | 2.5 | 10 | 40 | 80 |
| 1 | 40 | 64 | 81 | 93 | ND | 31 | 60 | 81 | 94 | ND |
| 1K | 47 | 78 | 91 | ND | ND | 36 | 67 | 86 | ND | ND |
| 2 | 47 | 75 | 91 | ND | ND | 39 | 74 | 91 | ND | ND |
| 2K | 26 | 63 | 84 | ND | ND | 29 | 65 | 86 | ND | ND |
| 3 | ND | ND | 93 | 98 | 101 | ND | ND | 22 | 68 | 94 |

ND: not determined

The activity profile of Compound 2 is substantially higher than that of Compound 1. The salification of Compounds 1 and 2 has opposite effects in terms of activity in this model.

| | Dose | Plasma 1 h | | Plasma 4 h | |
|---|---|---|---|---|---|
| | (mg/kg) | (ng/ml) | % inh. | (ng/ml) | % inh. |
| Com- | 2.5 | 4237 ± 1260 | 75 ± 4 | 3472 ± 2433 | 63 ± 6 |
| pound 1K | 10 | 13900 ± 2117 | 89 ± 1 | 6813 ± 674 | 85 ± 2 |
| Com- | 2.5 | 2598 ± 1478 | 65 ± 8 | 2205 ± 1250 | 58 ± 5 |
| pound 1 | 10 | 3675 ± 1781 | 75 ± 8 | 3823 ± 2011 | 73 ± 4 |

Inh.: inhibition.

Compound 1 is detected in the plasma of the treated mice and inhibits 11β-HSD type 1 as of 0.63 mg/kg. LC/MS analyses of plasma samples from the animals treated with Compound 1K show a much higher plasma level of the parent molecule than that found for Compound 1.

### EXAMPLE 3

### Evaluation of the Bioactivity of White Adipose Tissue After Single Oral Administration of Various 11β-HSD Type 1 Inhibitor Compounds in C57BL/6N Mice (Distribution/Power/Efficacy)

### Protocol

Four hours post-administration, the mice are euthanized and inguinal white adipose tissue is taken and frozen at −70° C. until the day of bioanalyses. The white adipose tissue is homogenized in liquid nitrogen and then treated with acetonitrile (1 ml of distilled water for 4 ml of acetonitrile and 200 mg of inguinal white adipose tissue) to extract soluble substances. The acetonitrile fraction is collected, then dried, and the residue is dissolved in 1 ml of DMSO for 300 mg of white adipose tissue. The bioactivity of the inguinal white adipose tissue (the equivalent of 150 μg of tissue per well, 1% DMSO) is analyzed by using the inhibition of human 11β-HSD type 1 as a detection system (SPA technology) for each compound and each dose tested.

Compounds tested: the same as those of Example 2.

### Results

| | % inhibition after 4 h | | | | |
|---|---|---|---|---|---|
| | Doses (mg/kg) | | | | |
| Compounds | 0.63 | 2.5 | 10 | 40 | 80 |
| 1 | 8 | 36 | 63 | 79 | ND |
| 1K | 21 | 41 | 70 | ND | ND |
| 2 | 24 | 58 | 82 | ND | ND |
| 2K | 17 | 46 | 72 | ND | ND |
| 3 | ND | ND | 15 | 8 | 13 |

ND: not determined

As in Example 2, Compound 2 is substantially more active than Compound 1 and salification produces opposite effects.

### EXAMPLE 4

### Inhibition of Conversion of Prednisone to Prednisolone After Single Administration of Compounds 1, 1K and 2K to Cynomolgus Monkeys (Power/Efficacy)

Protocol

The protocol used is described by Bhat et al. (J. Pharmacol. Exp. Ther. 324, 299-305, 2008). A primate was selected as the species, because the 11β-HSD enzyme shows a quite marked species specificity between rodents and humans.

Briefly, the study is carried out in naive adult male cynomolgus monkeys fasted overnight. The 11β-HSD type 1 inhibitor or its vehicle (0.5% methylcellulose in water) is administered via nasogastric intubation. Two hours later, a challenge with orally administered prednisone (10 mg/kg) is performed and blood samples are taken regularly for 24 hours. These samples are centrifuged to obtain plasma and immediately frozen at −70° C. until bioanalysis. Plasma levels of prednisone, prednisolone and 11β-HSD type 1 inhibitors are measured by LC-MS/MS analytical methods.

The study is carried out over a period of 10 weeks. Profiles of plasma concentration as a function of time and areas under the curve are calculated using the trapezoidal method for all parameters.

Three Studies were Performed:

Study no. 1

The effect of Compound 1 is evaluated at three doses: 1.25, 5 and 20 mg/kg.

The inhibition of 11β-HSD type 1 mediated by Compound 1 is characterized by a reduction of conversion of prednisone to prednisolone and reflects a predominant hepatic impact within this specific time period. The plasma prednisolone/prednisone ratio of the area under the curve calculated between 30 minutes and 4 hours is the key parameter for the evaluation of efficacy. In this first study, Compound 1 shows significant and dose-dependent inhibition of 11β-HSD type 1 as of 5 mg/kg, the inhibition reaching 34% at 20 mg/kg. Considering the interindividual variability of this study performed with monkeys, 80% of the animals treated with Compound 1 at 20 mg/kg respond favorably to the pharmacological treatment with a 46% inhibition of the prednisolone/prednisone ratio. Similarly, 60% of the animals respond favorably to a dose of 5 mg/kg of Compound 1, with inhibition reaching 28%. It should be stressed that the effect on adipose tissue, for which Compound 1 seems to have very good distribution as shown by the results in mice, is not addressed in this model due to the experiment's specific time frame. No particular clinical or behavioral sign was reported with this Compound 1 during this study.

Study no. 2

The effect of Compound 1K is evaluated at three doses: 20, 40 and 80 mg/kg. The protocol is quite similar to the preceding, except that only 5 blood samples were taken per monkey rather than 7. Compound 1K was also evaluated over a period of 10 weeks.

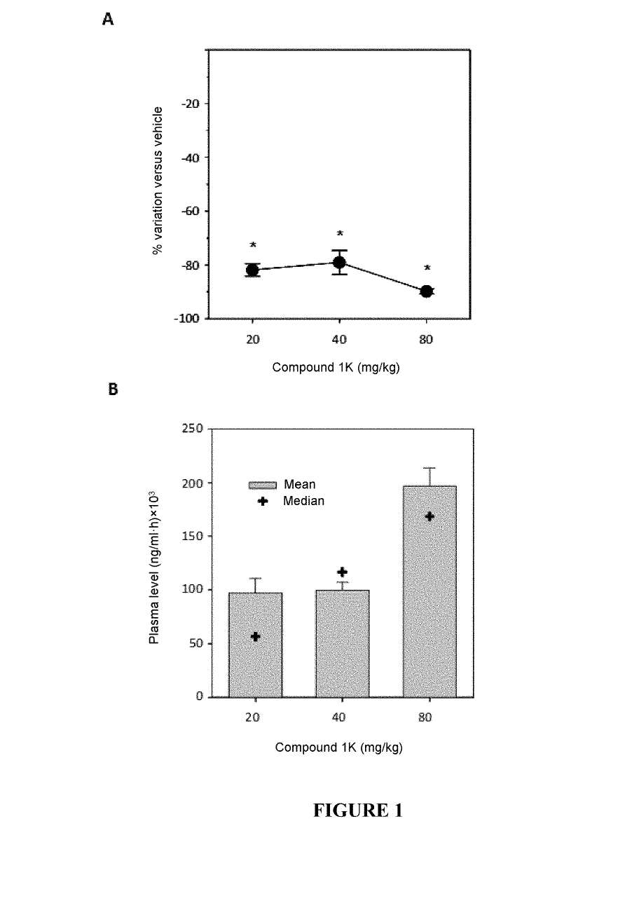

Conversion of prednisone to prednisolone is dependent on the activity of 11β-HSD type 1; Compound 1K induces a “maximum” inhibition (of at least 80%) of the plasma prednisolone/prednisone ratio of the area under the curve calculated between 30 minutes and 4 hours, and this result is obtained for all the doses tested (FIG. 1A), with 5 animals per group; the pharmacological response has low interindividual variability. FIG. 1 represents the effects of Compound 1K on the plasma prednisolone/prednisone ratio, calculated with the area under the curve between 30 minutes and 4 hours (panel A) and plasma exposure for the 6-hour experimental period (panel B).

At the same dose of 20 mg/kg, the potassium salt (1K) and the non-salified form (Compound 1) inhibit this biomarker by 81% and 34%, respectively. This major difference in pharmacological activity is likely related to plasma exposure, which increases nearly 8-fold with Compound 1K. This protocol makes it possible to evaluate chiefly the activity of hepatic 11β-HSD type 1. At the highest doses of 40 and 80 mg/kg, plasma exposure of Compound 1K further increases (FIG. 1B), and conversion of prednisone to prednisolone is significantly inhibited by 79% and 89%, respectively. No particular clinical or behavioral sign was reported with Compound 1K during this study, even at 80 mg/kg.

Study no. 3

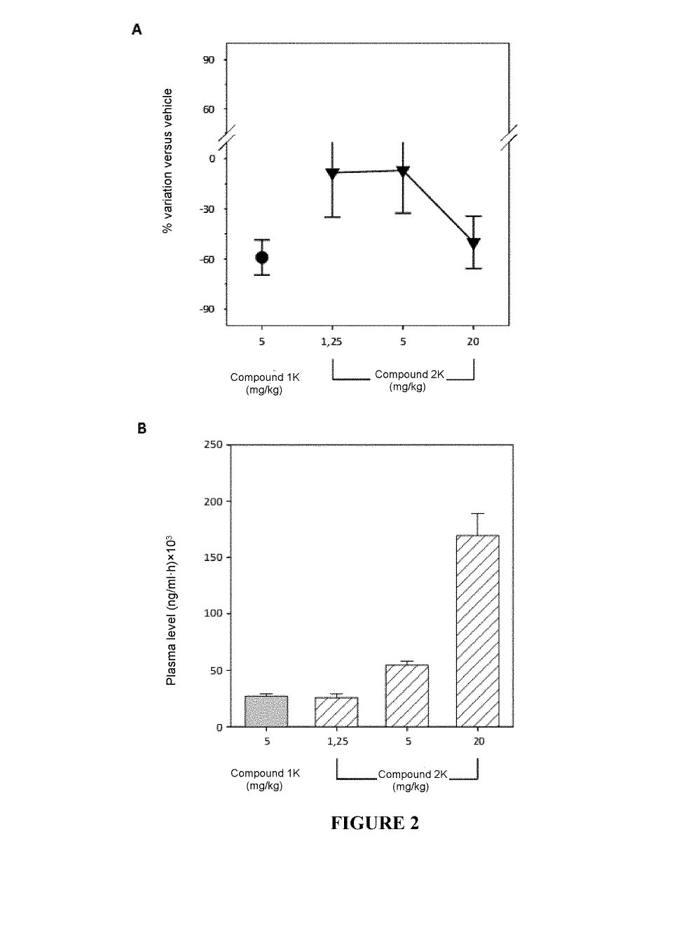

An experimental protocol comparable to the preceding was carried out on 5 naive primates to test the dose-response relationship (1.25, 5 and 20 mg/kg) of Compound 2K, and for comparison one dose of 1K (5 mg/kg). The 11β-HSD type 1 activity-dependent conversion of prednisone to prednisolone was evaluated. Compound 2K inhibits roughly 50% this activity at a dose of 20 mg/kg (FIG. 2A). FIG. 2 represents the effects of Compound 2K, at the three doses tested, on plasma prednisolone/prednisone ratio calculated with the area under the curve between 30 minutes and 4 hours (panel A) and on plasma exposure during the 6-hour experimental period (panel B); the results obtained with Compound 1K at a dose of 5 mg/kg are compared in the two panels. This study shows that Compound 2K at doses below 20 mg/kg is inactive; it is surprisingly less powerful than Compound 1K even though its plasma exposure is higher (FIG. 2B). However, this much less favorable activity profile for Compound 2K is not due to an intrinsic power to inhibit 11β-HSD type 1 inferior to Compound 1K, quite to the contrary (see Example 1).

### EXAMPLE 5

### Inhibition of Hepatic 11β-HSD Type 1 After Single Oral Administration of Compound 1K in Cynomolgus Monkeys (Ex Vivo Activity)

### Protocol

Five adult male cynomolgus monkeys (same animals used in studies 1 and 2 of Example 4) are fasted the evening before the experiment. Blood samples are taken right before administration of Compound 1K (dose of 20 mg/kg, n=3) or administration of the vehicle (0.5% methylcellulose in water, n=2) then 4 hours after oral administration. The animals are euthanized, necropsy is performed on the monkeys, samples of plasma and of target tissues are taken and then frozen in liquid nitrogen and stored at −70° C. until ex vivo analyses. To test the ex vivo activity of 11β-HSD type 1, 5-10 mg liver samples were incubated in duplicate for 30 minutes at 37° C. with 17 nM [1,2-³H] cortisone in 50 mM HEPES (pH 7.4), 100 mM KCl, 5 mM NaCl, 2 mM MgCl₂. Plasma levels were measured by the LC-MS/MS analytical method. The liver samples were homogenized by sonication and extracted with acetonitrile (1 ml of distilled water, 4 ml of acetonitrile for 200 mg of tissue). Tissue levels of the compounds were measured by the LC-MS/MS analytical method.

### Results

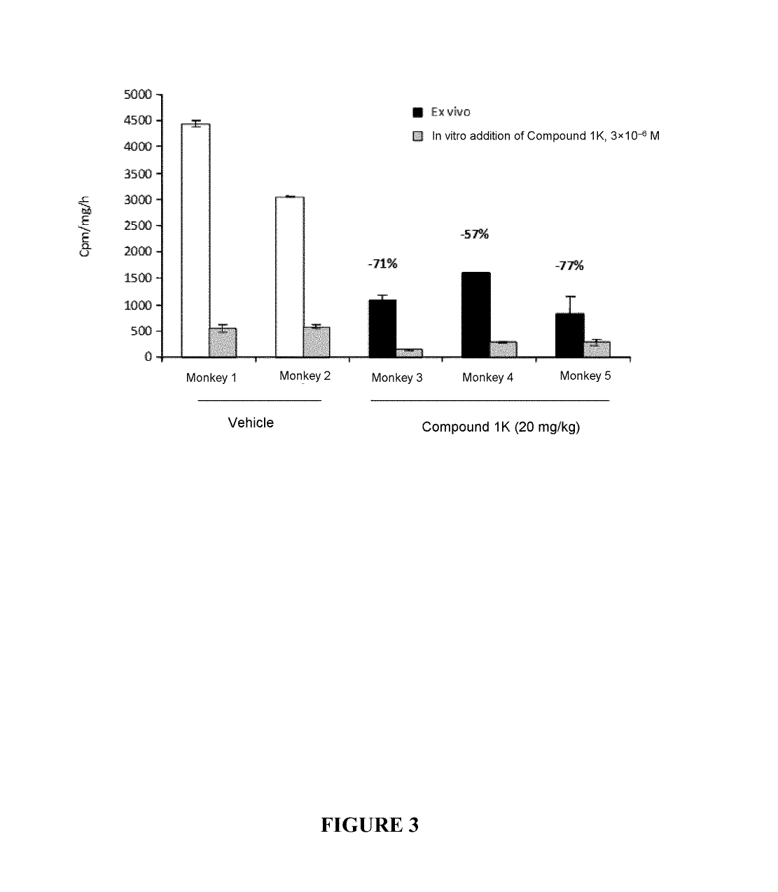

Four hours after oral administration, mean plasma levels of Compound 1K were 16967±3576 ng/ml (SEM); these data are in perfect harmony with the results of the previous studies. FIG. 3 shows the effects of administration of Compound 1K at 20 mg/kg on ex vivo activity of 11β-HSD type 1 in the liver of cynomolgus monkeys.

Single oral administration of the compound results in a substantial inhibition of 68% of the ex vivo activity of hepatic 11β-HSD type 1, which may be further inhibited by in vitro addition of 3×10⁻⁶ M Compound 1K (FIG. 3). Mean hepatic levels (in relative value) of Compound 1K are 13.6±3.7 ng/mg tissue.

### EXAMPLE 6

### Inhibition of 11β-HSD Type 1 of Adipose Tissue (Mesenteric and Subcutaneous) After Single Oral Administration of Compound 1K to Cynomolgus Monkeys (Ex Vivo Activity)

### Protocol

The protocol is the same as that described in Example 5, except that the tissue samples are from mesenteric, subcutaneous and inguinal adipose tissue.

### Results

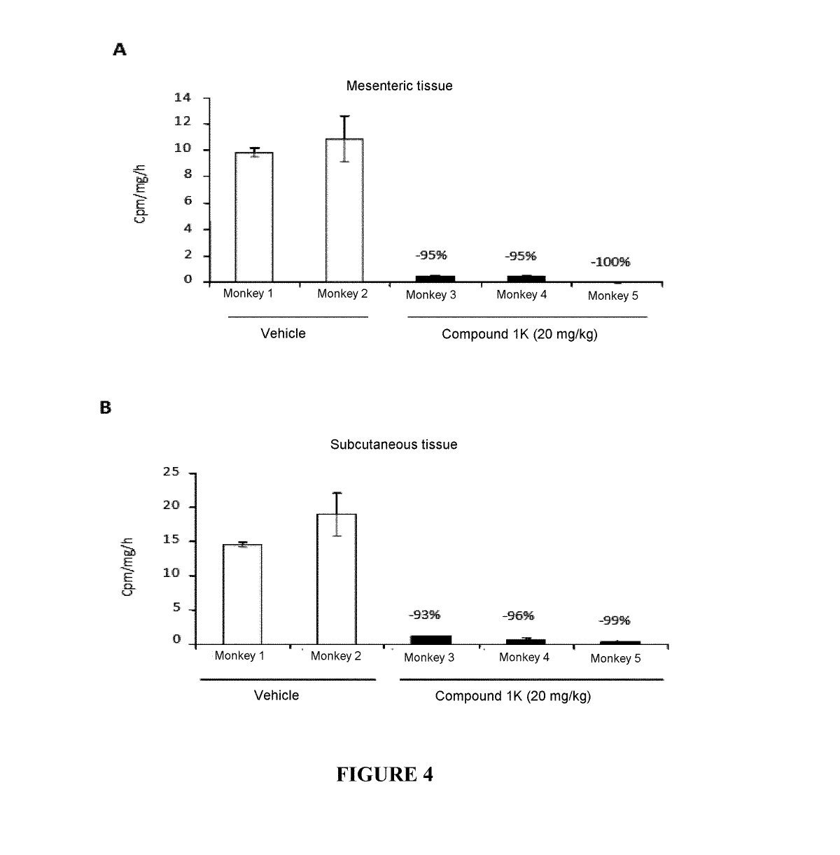

FIG. 4 summarizes the effects of administration of Compound 1 at 20 mg/kg on ex vivo activity of 11β-HSD type 1 in adipose tissue of cynomolgus monkeys. Single oral administration of Compound 1K induces complete inhibition of ex vivo activity of 11β-HSD type 1 of peripheral and visceral adipose tissue. The results on inguinal adipose tissue are similar (data not shown). Tissue levels (in relative value) of Compound 1K in mesenteric, subcutaneous and inguinal adipose tissues are 6.6±0.4, 1.3±0.1 and 6.1±1.5 ng/mg, respectively.

These results show the extremely strong tropism of Compound 1K to penetrate adipose tissue.

1.-7. (canceled)

8. A method for preventing and/or treating fatty liver comprising the administration to a patient in need thereof of an effective amount of (4-Hydroxy-2-methyl-1,1-dioxido-2H-benzo[e][1,2]thiazin-3-yl)(naphthalen-2-yl)methanone, or a pharmaceutically acceptable salt thereof.

9. The method according to claim 8, wherein (4-Hydroxy-2-methyl-1,1-dioxido-2H-benzo[e][1,2]thiazin-3-y)(naphthalen-2-yl)methanone is in the form of a pharmaceutically acceptable salt which is a potassium salt.

10. The method according to claim 8, wherein the patient is a patient with non-alcoholic steatohepatitis.

11. The method according to claim 8, wherein the patient is a patient with liver fibrosis, cirrhosis, liver failure or hepatocellular carcinoma.

12. The method according to claim 8, wherein the patient is a patient undergoing treatment for type 2 diabetes or dyslipidemia.

13. A method for preventing and/or treating fatty liver comprising the administration to a patient in need thereof of an effective amount of a pharmaceutical composition containing as active ingredient (4-hydroxy-2-methyl-1,1-dioxido-2H-benzo[e][1,2]thiazin-3-yl)(naphthalen-2-yl)methanone, or a pharmaceutically acceptable salt thereof, and at least one pharmaceutically acceptable excipient.

14. The method according to claim 13, wherein (4-Hydroxy-2-methyl-1,1-dioxido-2H-benzo[e][1,2]thiazin-3-yl)(naphthalen-2-yl)methanone is in the form of a pharmaceutically acceptable salt which is a potassium salt

15. The method according to claim 13, wherein the patient is a patient with non-alcoholic steatohepatitis.

16. The method according to claim 13, wherein the patient is a patient undergoing treatment for type 2 diabetes or dyslipidemia.

17. The method according to claim 13, wherein the patient is a patient with liver fibrosis, cirrhosis, liver failure or hepatocellular carcinoma.
